(19) United States
(12) Patent Application Publication
(10) Pub. No.: US 2026/0174945 A1
(43) Pub. Date: Jun. 25, 2026

(54) TREATMENT OF ALZHEIMER'S DISEASE

(71) Applicant: ISAAC ELIAZ, Santa Rosa, CA (US)

(72) Inventor: ISAAC ELIAZ, Santa Rosa, CA (US)

(21) Appl. No.: 18/991,523

(22) Filed: Dec. 21, 2024

(51) Int. Cl. A61M 1/34; A61K 31/732

(52) U.S. Cl. CPC A61M 1/3496; A61K 31/732; A61M 2202/07

(57) ABSTRACT

Patients suffering from, or at risk of developing the symptoms of, Alzheimer's disease and related neuroinflammatory-based diseases, are treated by apheresis to selectively withdrawal Galectin-3 from the patient's body. A reduction in the circulating level of gal-3 of the patient of at least 30% of the patient's pre-treatment galectin-3 level should be sufficient to reduce AD symptoms, and/or inhibit AD progression. A greater withdrawal, up to at least 20%, has greater impact. Selective withdrawal of Galectin-3 may be coupled with the administration of MCP to further slow the progress of, and/or reverse, AD symptoms.

### PRIORITY DATA AND INCORPORATION BY REFERENCE

This application is related to, but does not claim benefit of priority from, U.S. patent application Ser. No. 18/817,781 (hereinafter “the '781 application”) directed to the treatment of Sepsis and Acute Kidney disease, and related conditions, through apheresis. Since both the '781 application and this application are directed to treatment of conditions in a mammal, including humans, through apheresis to effect a reduction in Gal-3 levels, the disclosure of that earlier application is incorporated herein by reference.

### BACKGROUND OF THE INVENTION

### Field of the Invention

The present invention relates to the treatment, and possible prevention, of Alzheimer's disease and other neurodegenerative conditions mediated by accumulation of pathological proteins and neuroinflammation, including frontotemporal dementia, progressive supranuclear palsy, amyotrophic lateral sclerosis, corticobasal degeneration, dementia, and Parkinson's disease. A similar neuroinflammatory response is seen in patients experiencing Transient Ischemic Attacks and Cerebro Vascular Accidents (CVA), where the damage may arise simply from conditions arising from a lack of oxygen ot the brain. In the context of this invention, addressing the body's response to brain hypoxia, whether confined solely to Alzheimer's disease, or also arising from other situations caused by the Gal-3 driven conditions, treatment lies in part in apheresis to effect the selective withdrawal of Gal-3 from the body. Alzheimer's disease and related conditions as described, referred to throughout this application as “AD”, presents an issue for medical research and application as it is exhibited only in humans, and model animals like mice and similar experimental models prepared from genetic engineering techniques. The treatment set forth herein relies on apheresis to draw blood through an ex vivo treatment system first disclosed in U.S. Pat. No. 10,953,148. In that system, blood is withdrawn from the patient, and may be separated into plasma on the one hand and blood cells on the other, with the plasma passed to one or more conduits where the blood is treated. As disclosed in U.S. patent application Ser. No. 17/964,644, the entire disclosure of which is incorporated herein by reference, the same apparatus may be used for effective apheresis using whole blood, avoiding the need to separate the blood into plasma and blood cell components.

### SUMMARY OF THE INVENTION

Gal-3 plays a multifaceted role in the pathogenesis and progression of AD and other dementia, acting through various mechanisms that influence neuroinflammation, Aβ dynamics, and neuronal network function. In AD, Gal-3 is implicated in the promotion of Aβ oligomerization, a process associated with neurotoxicity, suggesting that targeting Gal-3 could be a potential therapeutic strategy to mitigate the formation of neurotoxic oligomers. Applicant has determined that apheresis to effect the selective withdrawal of Gal-3 in adult humans may have far-reaching effects. It is effective in the prevention of the development of dementia and related symptoms in adults likely to develop dementia or exhibit aspects of dementia. Apheresis to achieve selective withdrawal of Gal-3 in adults exhibiting dementia is similarly likely to lead to reduction or elimination of symptoms associated with dementia. Remarkably, human patients exhibiting profound dementia symptoms may also be treated using apheresis to effect selective withdrawal of Gal-3 in order to improve and/or reverse the symptoms of dementia in those already identified and diagnosed as suffering from AD.

This is further supported by findings that Gal-3 is involved in microglial activation, a key component of the neuroinflammatory response in AD, where it is primarily expressed by microglial cells clustered around Aβ plaques. The deleterious role of Gal-3 in microglial activation and its association with larger and more irregular amyloid plaques and neurons containing tau inclusions highlight its contribution to AD pathology. It is also likely associated with the neuroinflammatory response to accumulation of other pathological proteins, including, but not limited to, transactive response DNA binding protein 43 kDa (TDP-43), fused-in-sarcoma (FUS) protein, Ewing sarcoma breakpoint region 1 (EWSR1 gene encoding the EWS protein) and TBP associated factor 15 (TAF15).

Moreover, Gal-3 has been shown to impair neuronal network dynamics, specifically by disrupting gamma oscillation power and rhythmicity, which is crucial for cognitive function. This disruption is mediated through the spike-phase uncoupling of fast-spiking interneurons, leading to a collapse in network performance. The involvement of Gal-3 in neuroinflammation is further evidenced by its role in regulating key inflammatory pathways, such as the TLR4/NF-κB pathway, suggesting that Gal-3 inhibition could alleviate neuroinflammation and neuronal apoptosis, thereby improving cognitive function. Gal-3 have been found to be higher in AD patients compared to controls, suggesting its utility as a biomarker for the disease. Collectively, these findings underscore the complex role of Gal-3 in AD, involving neuroinflammation, Aβ dynamics, and neuronal network function, making it an effective target for therapeutic intervention. Collectively, these data strongly indicate that selective removal of at least 30% of gal-3 across the apheresis column (reduction of at least 30% between Gal-3 concentration in the blood/plasma above/prior to the entrance to the column and below/after exiting the column) via apheresis may not only reduce or slow the progress of AD, but reverse or limit its effects in many patients.

Therefore, Gal-3 not only activate microglia to chronically skew in M1 phase (via TREM and TLR4) but escalate AD pathophysiology in a vicious cycle as it contributes to Aβ oligomerization and to the deficiency of several enzymes that are part of the normal degradation of Aβ such as IDE-1, NEP, Transthyretin (either by regulation or binding) and may affect the phagocytosis pathway. Last but not least, Gal-3 inhibits integrin-α3-mediated signaling which may results in memory impairment.

Gal-3 binds glycoprotein oligosaccharides through its carbohydrate recognition domain (CRD), which mediates its pharmacological inhibition by blocking the protein's activity. Different inhibitors with different types of cellular uptake have been synthesized which may affect the Gal-3 activity with regard to its localizations in tissues, (intracellular, membrane-bound or extracellular), with implications of it its effect on the multiple hallmarks of AD. Gal-3 inhibition in the 5×FAD mouse with TD139 (a 3,3′-bis-(4-aryltriazol-1-yl) thio-digalactoside Gal-3 inhibitor with high affinity for the Gal-3 carbohydrate recognition domain prevented the oligomerization Aβ42 which suggests that Gal-3 supports plaque formation by its cross linking between carbohydrate groups. Adding 1,1′-sulfanediyl-bis-3-deoxy-3-4-3-fluorophenyl-1H-1,2,3-triazol-1-yl-β-d-galactopyranoside (another Gal-3 inhibitor) resulted with significantly reduced release of proinflammatory cytokines TNFα, IL12 and IL8 in vitro (BV-2 cells). In this study gal-3 inhibition, upregulated IDE-1 also supported by IDE-1 levels which were increased in 5×FAD/Gal3KO mice compared to 5×FAD mice. TB006, a monoclonal antibody targeting Gal-3 tested in three AD mouse models acts as a possible treatment for AD by degrading neurotoxic Ab oligomers. MCP helped protect the blood-brain barrier and reduced brain injury in a mouse model of subarachnoid hemorrhage, suggesting that Gal-3 contributes to brain injury by regulating inflammatory responses. Modified Pectin (often referred to as Modified Citrus Pectin or MCP) also reduced neuroinflammation, oxidative stress, and cognitive impairment in diabetic rats induced by a high-fat diet and streptozotocin (HFD/STZ). Additionally, MCP lessened inflammation and oxidative stress in BV-2 microglial cells stimulated with high glucose. Other Gal-3 binding agents may be administered in the same fashion that MCP is provided. Overall, these studies indicate that Gal-3 inhibition can protect neurons from inflammation-induced damage, particularly in the hippocampus and striatum. Other effective agents of this nature that may be administered include synthesized carbohydrates, antibodies to Gal-3 and pectin fragments.

### Adsorption of Gal-3 Porcine Study

As set forth in U.S. patent application Ser. No. 18/827,781 filed Sep. 8, 2024, using the XGAL-3® column to deplete Gal-3 in LPS-induced sepsis porcine model, 2,357 genes and 268 proteins were down regulated and 1,826 genes and 131 proteins were upregulated. Downregulation of inflammation/neuroinflammation signaling pathways may also attenuate the AD exacerbation.

Interestingly, depletion of Gal-3 upregulated the occludin, ZO-1 which are the membrane-associated cytoplasmic proteins in BBB TJs. Compromise of the BBB integrity is one of the hallmarks in AD and the depletion of GAL-3 is suggested to contribute to the recovery of the BBB. The XGAL-3 column was able to stabilize the capillary leak and support the integrity of the tight junctions. The XGAL-3 column refers to an apheresis conduit provided with an antibody which binds gal-3 such that when the conduit receives blood, any galectin-3 is bound, while the remaining blood is returned to the patient.

Gal-3 adsorption was found to down regulate PI3K-AKT and HIF-1 signaling pathways which are known to be involved in the phosphorylation of APP and hyper phosphorylation of Tau (by GSK3) causing the aggregation of SPs and NFT through the neuroinflammation process (both astrocytes and microglia activation). Therefore, we can expect to block or halt in both pathophysiology generation processes.

Gal-3 activates microglia through pathways involving IFN-γ, TLR4, and IGFR-1, promoting proinflammatory responses and microglial proliferation, while also supporting phagocytosis by binding MerTK after sialidase secretion. Additionally, Gal-3 binds LPS to inhibit inflammatory signaling and interacts with AGEs to detoxify them through lysosomal fusion.

Applicant has determined that effective treatment of AD in mammals, including humans, can be achieved by selective withdrawal of Gal-3 through apheresis. This ex vivo treatment of blood, to lower the concentration of Galectin-3 (GAL-3) has been shown to effectively impact patients which should lead to improved AD outcomes. The aforementioned neurodegenerative conditions are largely mediated by the inflammatory response to accumulation of pathological proteins. By preventing and ameliorating the neuroinflammatory response, the process of neurodegeneration can be prevented and treated. As a result, Gal-3 removal may be utilized to prevent neuroinflammation as soon as elevated levels of pathological proteins and/or their downstream effects are detected, including Tau, TDP-43, FUS, EWS, TAF15, amyloid beta plaques, and neurofibrillary tangles. Gal-3 activates the neuroglia that likely mediate the neuroinflammatory to these proteins.

### DETAILED DESCRIPTION OF THE INVENTION

To effectively address AD, a patient is treated with apheresis. There are a wide number of apheresis protocols and treatments known, for various purposes. To effectively treat patients to limit or reverse AD, the apheresis may be of blood (plasma) separated into separate portions, so as to remove one or more of white blood cells, platelets, red blood cells and plasma. Details and protocols for apheresis vary widely, but are neatly summarized and detailed in U.S. Pat. No. 10,953,148, assigned to Eliaz Therapeutics, Inc. of California. Recently, the same researchers have made strides in apheresis treatment using whole blood apheresis, as set forth in detail in U.S. patent application Ser. No. 17/964,644. Thus, it is also possible now to treat certain cases with whole blood apheresis and/or plasmapheresis, depending on targets and conditions. The disclosure of both U.S. Pat. No. 10,953,148 and U.S. patent application Ser. No. 17/964,644 are incorporated herein-by-reference in their entirety. Importantly, the treatment may be used in conjunction with immunotherapies, including but not limited to lecanemab and donanemab.

Apheresis is a technique developed to treat a variety of conditions and pathologies. Many of these treatments are effected by removing one type of blood, one agent or one component of the blood, and returning the remainder to the patient. Whether the apheresis is of whole blood or the more conventional treatment following separation the target in this invention for the treatment of AD is Galectin-3, referred to herein as Gal-3.

Gal-3 has been shown to be an upstream mediator of the inflammatory cascade underlying AD. By intervening to lower levels of Gal-3 in the blood by at least about 30%, through selective withdrawal treatment of AD patients may be effective in improving and even reversing their condition. This process of using apheresis to remove a component of blood, in this case gal-3, has come to be referred to as “selective withdrawal” of the target. To treat AD, significant levels of selective withdrawal of Gal-3 may be required. Selective withdrawal of more than 50% of the patient's Gal-3 content may be required. The selective withdrawal of Gal-3 to treat dementia may require withdrawal of up to 60, 70 and even 80% of the patient's pre-apheresis Gal-3 concentration. Thi treatment is effective to reduce a patient's Gal-3 level to values at or below the patient's pre-apheresis Gal-3 levels. As those of skill in the art will appreciate, to effectively reverse dementia symptoms, or otherwise maintain a patient's condition, apheresis may be repeated periodically, at levels sufficient to reduce or eliminate key dementia symptoms. As ongoing monitoring is required, apheresis may be repeated when necessary, but in general, at least every 14 days is a reliable guidance. Initially, more frequent apheresis treatments may be required, including the possibility of apheresis each day for the first three days. For certain individuals, less frequent removal can still be beneficial. Of importance, in the context of dementia, once the inflammation is reduced to normal levels following apheresis, Gal-3 levels systemically may come down on an on going basis. One may see a systemic reduction right away after the treatment of those exhibiting symptoms reflecting AD related dementia that persists for some time.

Average Gal-3 blood levels in healthy adults typically range from 8 to 15 ng/ml. In patients with AD, Gal-3 levels are usually significantly elevated beyond this range. The effectiveness of apheresis in reducing Gal-3 levels in AD patients can vary; while selective withdrawal of Galectin-3 through apheresis will result in a notable decrease across the column, the levels of Gal-3 in the blood may not reflect it in the initial phase, as patients will likely continue to produce excess Gal-3 during the initial period of their treatment. Generally, Gal-3 apheresis treatments in AD patients should be administered for an initial period of 3-5 consecutive days, depending on the patient's Gal-3 levels and clinical outcome after initial treatment, followed by periodical treatments every 7-14 days, based on Gal-3 rebound rate and clinical improvement. Additional treatments may be considered based on the patient's Gal-3 levels and clinical response after the initial treatment series. The invention remarkably has demonstrated effective results for some patients following a single day of treatment.

Selective withdrawal is effected by passing the patient's blood in the apheresis device over an array of binding agents secured to the apheresis device interior. The binding agents are typically anti-bodies, either natural or recombinant, that effectively bind to galectin-3. The Gal-3 so bound stays in the apheresis tube while the blood is passed through and thereafter returned to the patient. The binding agent may be a natural protein and the like if the binding coefficient is high enough. The term selective withdrawal has developed over the last few years to distinguish the apheresis process described here, where a particular target, like Gal-3, is withdrawn from the blood selectively, as opposed to a process where blood is passed through or over a filter that captures a portion of the blood based on, e.g., molecular weight. Various references to such filters appear in the art, such as in U.S. Patent Publication 2023/0107479, Mutandum, or Patent Publication 2021/0388065 to Lu.

In contrast, the claimed invention relies on selective withdrawal, where the blood, following fractionation if desired into plasma and blood cells, is passed through at least one conduit. The interior of the conduit has affixed to it elements that will bind the target of the apheresis, in this case, for example, an antibody or similar binding fragment that binds to Galectin-3. As the blood is passed, ex vivo, through the conduit under the influence of a pump, it comes into contact with the binding agent, which is secured to the interior of the conduit by a permanent bonding such as Protein A or Sepharose links. As the blood drawn from the patient's body passes through the conduit, the target compound, in this case Galectin-3, is bound by the exposed antibody, and remains, while the blood passes along and then is returned to the body. The returned blood, clearly, has a lower concentration of Gal-3 than the patient exhibited prior to apheresis. In this process, repeated apheresis treatments can provide an effective reduction of Gal-3, providing a treatment for Alzheimer's disease not previously available.

It is of course clear that a given patient may be treated by selective withdrawal of more than one target. While conduits may be lined with multiple binding agents to secure binding of more than one target in a single conduit—better results have been secured by simply passing the blood ex vivo through more than one conduit, each conduit lined internally with an agent that binds the target. The invention contemplates that in addition to the selective withdrawal of galectin-3 in the treatment of AD, other targets may be made the subject of selective withdrawal for the same patient. A detailed description of a conduit provided with a binder that is an antibody is provided in this application to further illustrate the technology. Thus, in addition to selective withdrawal/removal of Gal-3, the invention may further comprises the selective withdrawal of other targets, including inflammatory compounds, cytokines and LPS via whole blood or plasma apheresis. The invention specifically contemplates, in addition to removal of Gal-3, the selective withdrawal of LPS, CRP, different cytokines, and microbial agents (whole blood) including withdrawal of specific targets including lipopolysaccharides (LPS) C-Reactive Protein, IL-4, IL-6, IL-1B, IL-10, TNF alpha, NF-κB, High Mobility Group Protein B1 (HMGB1) and combinations thereof. More generally, other cytokines may also be addressed by selective withdrawal, including various chemokines, interferons, other interleukins and the like. These include without limitation Interleukin-1 (IL-1), Interleukin-10 (IL-10), Interleukin-8 (IL-8), Interleukin-12 (IL-12), Interferon-gamma (IFN-γ), Monocyte Chemoattractant Protein-1 (MCP-1/CCL2), Transforming Growth Factor-beta (TGF-β) and Interleukin-17 (IL-17). These and similar agents are discussed in www.sinobiological.com/resource/cytokines/role-of-cytokines-in-sepsis.

As also described in patents similar to U.S. Pat. No. 10,953,148 and many other texts, effective treatment may depend not only on selective removal of gal-3 from the blood, and other blood agents, but it may depend on the administration or addition of therapeutic or beneficial elements before the blood is returned to the mammal. Often, addition of the agent to the plasma is an effective and efficient means of administration of the treatment, such as a medication, vitamin, or similar component. Thus, while not essential to the selective removal of gal-3, the methods set forth in this disclosure include administration of a variety of agents to the patient, which may be effectively achieved by addition to plasma and/or whole blood during the practice of apheresis. Examples include various homeopathic medications, metabolism regulators, immune reaction modifiers, pharmaceuticals, biological agents and various cytotoxic compounds, anti-microbial agents, chelating agents, possibly targeted ones, to aid in treatment.

### Apheresis Column Description

The XGal-3® column or conduit is a selective therapeutic apheresis column developed to selectively and specifically bind and remove Gal-3 from plasma by affinity adsorption. It thus is of specific value in the invention set forth herein for the treatment of AD. The XGal-3® column contains an immobilized high-affinity and highly-selective binding agent for the target molecule. As a selective extracorporeal apheresis technique, affinity adsorption has the clinical advantage of not depleting essential components of the blood and not requiring replacement fluid, electrolytes, and albumin. Unlike non-selective apheresis and Total Plasma Exchange (TPE), selective withdrawal targets a single molecule rather than depleting multiple nutrients and proteins, such as albumin and immunoglobulins (Ig), thus potentially offering a safer and more targeted therapeutic tool. The column developed for withdrawal of galectin-3, the XGal-3® apheresis column, can be effectively used in this invention to remove Gal-3 so as to treat AD.

The sterile, single-use XGal-3® column is connected into the plasma circuit of the apheresis system using standard tubing sets provided by Terumo BCT, the manufacturer of the apheresis system. The apheresis procedure is then performed in accordance with specific requirements of the apheresis protocol. This invention can be performed using any number of apheresis columns, but to demonstrate the invention clearly, the XGal-3 Column® is discussed here.

In this invention, a patient is first provided with the system, including the column through which the blood will pass, the plasma separator if employed, a pump, blood tubing and related elements. The patient's blood is removed via venous access. For many applications, the patient's blood is separated into plasma and cell fractions by an FDA-approved plasma separator. The plasma fraction is pumped through the XGal-3® column. As related in pending patent application U.S. Ser. No. 17/964,644, whole blood may be used for apheresis. The entirety of U.S. patent application Ser. No. 17/964,644 is incorporated herein-by-reference. Within the XGal-3 Column®, plasma is channeled through a specialized affinity matrix that captures Gal-3 molecules present in the plasma, binding them to the matrix structure. After passing through the XGal-3® column, the plasma is recombined with the cellular fraction, and the whole blood, now depleted of Gal-3, is returned to the patient intravenously.

The XGal-3® column housing is composed of a polycarbonate body (FIG. 1-2) filled with a mixture of 6% cross-linked agarose beads (Purolite, UK) suspended in phosphate-buffered saline (PBS) and 20% ethanol. The Gal-3 affinity ligand is a purified chimeric IgG1 antibody, derived from the CHO-K1 (Chinese Hamster Ovary) cell line, which is covalently bound to the agarose beads and specifically designed for high-affinity binding to Gal-3. The antibody purification process involves multiple chromatography steps, including ion exchange, affinity chromatography, and size exclusion chromatography (SEC-HPLC). This rigorous purification sequence ensures the production of an antibody with an endotoxin level of less than 0.04 EU/mg and high purity. The purity was confirmed by SEC-HPLC, which displayed a single, well-defined pauci-disperse peak with no detectable aggregates or higher-order structures. The human Fc chimeric antibody was chemically conjugated to the agarose resin through a three-multi-step process. First, functional groups on the agarose were activated to cross-link with a linker containing epoxy groups. The linker was then activated in a second step to facilitate conjugation with the chimeric antibody. The final step involved the deactivation of all remaining epoxy groups to prevent non-specific binding. This method achieved an antibody immobilization efficiency of greater than 95%, providing a highly stable resin. The resin was stored in 20% ethanol/DDW at 4° C. to maintain its activity. The beads are packed into the polycarbonate column, which is fitted with semipermeable polymer membranes at the top and bottom to retain the beads while allowing plasma to flow through. The column is equipped with ports compatible with standard apheresis device tubing and Luer-lock connectors, enabling the processing of plasma for Gal-3 removal.

When used as described, the XGal-3 column effectively depletes Gal-3, with preclinical studies discussed in the application showing high effectiveness and promising clinical benefits in removing Gal-3 from human plasma. Trials achieved nearly 100% efficiency of Gal-3 removal, with no detectable Gal-3 downstream of the column and no indication of capacity saturation, even when processing large plasma volumes. Validation studies have further confirmed the XGal-3 column's consistent and reliable performance in depleting Gal-3 across different residence time and plasma volumes. The XGal-3 column was tested for Gal-3 depletion using human plasma in an open one way system and a closed recirculated ex vivo system. The closed system was tested with a 40-second residence time and achieved near 100% depletion across the column and continuous depletion in the circulating plasma. The open system, tested at an 80 seconds residence time, maintained near 100% efficiency with no Gal-3 detected downstream of the column over a high plasma-to-column volume ratio, equivalent to 4 times the average plasma volume in an 80-kilogram patient. The results confirmed the XGal-3 Column® high efficiency, capacity, and suitability for clinical use.

The conjugation of the antibody used in the XGal-3 Column® discussed herein was facilitated by several chemical cross-linking steps: The agarose resin is activated with epichlorohydrin prior to dextran coupling. The addition of the dextran linker increases the surface area to conjugate the antibody. Then the dextran coupled agarose bead is activated with 1,4-butanediol diglycidyl ether, before attaching the antibody. To evaluate the conjugation efficacy of the antibody to the resin the absorbance at 280 nm of the antibody solution was recorded before and after the coupling. For instance, the absorbance values (AU280 nm) for the last batch were before coupling=0.6557 and after the coupling it went down to 0.012 which indicate 99.64% conjugation efficiency. The efficiency we require is greater than 95% to proceed for any procedure. The last step is to deactivate any remaining epoxy groups with tris-base. The final resin is stored in a 20% ethanol solution in DI water at 4° C. and filled into the column and equilibrated with phosphate saline buffer (pH 7.2) containing 20% ethanol.

The affinity of the mAb employed, 753B, to Gal-3 was confirmed by using the Biacore system. Biacore is a real-time biomolecular interaction analysis system based on surface plasmon resonance (SPR) technology which immobilizes the antibody and has the advantage over strategy of immobilizing antigens as ligands that only capture specific antibodies as it discriminates different epitopes and avoids the complex regeneration step. To further characterize the nature of the purified 753B, the purified antibody preparation was subjected to size-exclusion chromatography (SEC) to ascertain the presence of dimers, multimers, or aggregates (FIG. 5-13). The SEC chromatogram showed only a single pauci-disperse peak eluting at ˜7.723 minutes corresponding to the elution position of a standard human IgG1 molecule. No higher MW species were observed, suggesting the sample is substantially free of any aggregates or higher-order structures.

The adsorption efficiency of the XGal-3® column for Gal-3 was optimized by evaluating bead size and antibody density on the conjugated beads. A 2 ml mini-column packed with the Xgal-3 antibody-conjugated beads was tested using 60 column volumes of human plasma in a closed loop system. The plasma was re-circulated twice at the flow rate of 3 ml/min providing a residence time of 40 sec in the column. Plasma was applied to columns from a tank having a rotating magnetic bead and post-column effluent was recirculated to the plasma tank in a closed loop. Samples were periodically collected from the tank, as well as before and after the column, to measure the continuous Gal-3 depletion and the Gal-3 removal efficiency across the column.

The bead matrix conjugated with the antibody used in the most current column consistently achieved close to 100% Gal-3 depletion, with post-column Gal-3 levels dropping to undetectable amounts at a residence time of 40 seconds. There was no saturation or decline in depletion efficiency, even at a plasma-to-column volume ratio of 60:1 (equivalent to 3 liters of plasma in the XGal-3 column). Subsequent tests simulated the clinical flow rate scenario with a residence time of 80 seconds (equivalent to plasma flow of 37.5 ml/minute) using a high plasma-to-column volume ratio of 240:1 (equivalent to 12 liters of plasma with the 50 ml XGal-3 column) in an open system. The results confirmed nearly 100% Gal-3 depletion throughout the experiment, with no detectable levels of Gal-3 after the column, as well as no saturation. These findings indicate that the column has sufficient capacity for large-volume plasma processing without compromising its depletion efficiency.

Applicant has provided detailed information here as to the deployment and use of a specific apheresis column device referred to as the XGal-3 apheresis Column®. Other apheresis columns are known to those of skill in the art and can be used in place of the XGal-3 Column®, if desired. Applicant's invention has been developed specifically for the treatment of AD by selective withdrawal of galectin-3. The XGal-3 column, as described hereinabove, has been adapted for effective selective withdrawal of Gal-3. Other columns may be similarly developed for, and employed in this invention, to achieve effective reduction in Gal-3 levels.

While the present invention has been disclosed both generically, and with reference to specific alternatives, those alternatives are not intended to be limiting unless reflected in the claims set forth below. The invention is limited only by the provisions of the claims, and their equivalents, as would be recognized by one of skill in the art to which this application is directed.

What is claimed is:

1. A method of treating Alzheimer's disease in humans, comprising:
A) Identifying a human patient in need of treatment to address Alzheimer's disease, and
B) Treating said identified patient to selectively withdraw galectin-3 through apheresis so as to lower the patient's galectin-3 level by at least 30%

2. The method of claim 1, wherein said patient receives gal-3 selective withdrawal therapy so as to effect a reduction in said patient of at least 50% of galectin-3.

3. The method of claim 1, wherein said method further comprises administering to said patient modified pectin of no more than 40,000 Daltons.

4. The method of claim 1, wherein said patient is treated through selective withdrawal to withdraw at least 70% of the patient's galectin-3 across the column.

5. The method of claim 1, wherein said patient is likely to exhibit symptoms of dementia, and said apheresis treatment is effective to prevent said patient from exhibiting symptoms of dementia.

6. The method of claim 1, wherein said patient is exhibiting some symptoms of dementia, and said apheresis treatment is effective to prevent further deterioration in said patient resulting in the exhibition of additional symptoms of dementia.

7. The method of claim 1, wherein said patient is exhibiting symptoms of dementia, and said apheresis treatment is effective in reversing said patient's condition such that said patient exhibits fewer symptoms of dementia than prior to said apheresis treatment.

8. A method of treating a human patient suffering from disease mediated by neuroinflammation including any one of frontotemporal dementia, progressive supranuclear palsy, amyotrophic lateral sclerosis, corticobasal degeneration, and Parkinson's disease, comprising:
A) Identifying said patient and
B) Treating said identified patient to selectively withdraw galectin-3 through apheresis so as to lower the patient's overall galectin-3 level by at least 30%.
